(19) United States
(12) Patent Application Publication
(10) Pub. No.: US 2006/0257359 A1
(43) Pub. Date: Nov. 16, 2006

(54) Modifying macrophage phenotype for treatment of disease

(76) Inventors: Cedric Francois, Louisville, KY (US); Pascal Deschatelets, Louisville, KY (US); Paul Olson, Louisville, KY (US)

Correspondence Address: CHOATE, HALL & STEWART LLP, TWO INTERNATIONAL PLACE, BOSTON, MA, 02110, US

(21) Appl. No.: 11/364,670

(22) Filed: Feb. 28, 2006

Related U.S. Application Data
(60) Provisional application No. 60/656,992, filed on Feb. 28, 2005.

(51) Int. Cl. A61K 38/21; A61K 38/20; A61K 38/19

(52) U.S. Cl. 424/85.1; 424/450; 424/85.2; 424/85.4; 514/12

(57) ABSTRACT

The present invention provides compositions and methods for modulating one or more phenotypes of a macrophage-related cell, e.g., a macrophage. The invention further provides methods of treating disease by modulating macrophage phenotype. Representative phenotypes include pro-inflammatory, anti-inflammatory, immunogenic, tolerogenic, tissue-destructive, tissue restorative, cytotoxic, migratory, bone-resorbing, pro-angiogenic, anti-angiogenic, suppressor, antigen presentation, or phagocytic. Representative diseases include atherosclerosis, arthritis, and multiple sclerosis.

### CROSS-REFERENCE TO RELATED APPLICATION

This application claims priority to and the benefit of U.S. Provisional Patent Application Ser. No. 60/656,992, filed Feb. 28, 2005, which is incorporated herein by reference.

### BACKGROUND OF THE INVENTION

Macrophages are present in every tissue in the body and are part of the innate immune system. Macrophages are derived from myeloid precursors in bone marrow (BM), spleen, and fetal liver. Newly formed “inexperienced” macrophages, termed monocytes, leave the unique environment of the BM and enter the blood, where they are exposed to a plethora of agents, including cytokines, chemokines, adrenergic and cholinergic agonists, fatty acids, hormones, immunoglobulins (Igs), which are capable of affecting their functional and phenotypic characteristics. These monocytes selectively home to different tissues, presumably under the influence of chemokines or other tissue-specific homing factors. Upon entry into a tissue, the monocyte/macrophage migrates into the tissue parenchyma, the environment of which significantly influences the function of macrophages such that macrophages resident in different tissues display different patterns of function. Upon inflammatory insult to the tissue, these resident tissue macrophages can contribute to the innate immune response by expression of a variety of inflammatory and effector activities.

There has been substantial research activity in the past decade directed at phenotyping macrophage lineages and defining macrophage functional subsets or patterns of activity. The emphasis over the past 3-4 years has been to divide macrophage functional patterns into type 1 (Th1-driven) or type 2 (Th2-driven) functions. However, a huge array of environmental factors (including cytokines, chemokines, pattern recognition receptors, hormones) differentially regulates macrophage response patterns, resulting in the display of numerous distinct, functional phenotypes. In the art, the prevailing theory of macrophages is that they are end differentiated and not capable of transdifferentiation, i.e. macrophages are committed to a particular phenotype which cannot be changed.

While monocytes and more differentiated cells such as macrophages, osteoclasts, and dendritic cells fulfill a number of very important functions in the body, they can also contribute to the development and/or progression of a number of diseases and conditions. There is a need in the art for compositions and methods for treating these diseases and conditions.

### SUMMARY OF THE INVENTION

The present invention encompasses the recognition that phenotype(s) of macrophages and related cells can be modified, and provides compositions and methods for achieving such modification(s) for the treatment and/or prevention of a variety of diseases and conditions. These diseases and conditions, which include, but are not limited to, macular degeneration, atherosclerosis, osteoporosis, immune inflammation, non-immune inflammation, tuberculosis, multiple sclerosis, arthritis, chronic obstructive pulmonary disease (COPD), and Alzheimer's disease, may be caused or exacerbated, at least in part, by dendritic cells, monocytes, osteoclasts, or macrophages exhibiting one or more undesirable phenotypic characteristics or phenotypes. For example, in the case of arthritis, influencing macrophages in the affected joints to be more anti-inflammatory can alleviate the symptoms of the disease. Other conditions include transplant rejection, asthma, lupus, psoriasis, and scleroderma. In the case of certain diseases where macrophages or other macrophage-related cells are not thus far directly implicated, a change in macrophage or other macrophage-related cell phenotype can still alleviate the symptoms or the causes of the disease. The terms “modified” and “modulated” are used interchangeably herein.

In one aspect, the invention provides a method of modifying a phenotypic characteristic or phenotype of a macrophage-related cell in the body of a subject comprising administering an effector to the subject, wherein the effector is administered in an amount sufficient to cause a change in at least one phenotypic characteristic or phenotype of the macrophage-related cell. The macrophage-related cell may be selected from the group consisting of: dendritic cells, monocytes, osteoclasts, and macrophages, Administration of the effector optionally results in a change in the expression level of at least one gene in the macrophage-related cell. In certain embodiments of the invention the administering step comprises administering a composition containing one or more effectors and, optionally, one or more adjuvants, carriers, or excipients. In certain embodiments of the invention the phenotypic characteristic or phenotype is modulated in essentially all macrophage-related cells in the subject (e.g., at least 99% of the macrophage-related cells). In certain embodiments of the invention the phenotypic characteristic or phenotype is modulated in 20% of less, more than 20%, 40% or less, more than 40%, 60% or less, more than 60%, 80% of less, more than 80%, 90% or less, or more than 90% of all macrophage-related cells in the body. In certain embodiments of the invention the phenotypic characteristic or phenotype is modulated in 20% of less, more than 20%, 40% or less, more than 40%, 60% or less, more than 60%, 80% of less, more than 80%, 90% or less, or more than 90% of one or more types of macrophage-related cells in the body (e.g., any of the macrophage-related cell types mentioned herein).

In certain embodiments of the invention the effector agent is selected from the group consisting of: a cytokine, chemokine, pattern recognition receptor ligand, hormone, adrenergic and cholinergic agonist, fatty acid, phospholipid, immunoglobulin, Fc domain of immunoglobulins, lipopolysaccharide (LPS), toll-like receptor (TLR) ligand, histamine, peroxisome proliferator-activated receptor ligand, CD14 ligand, CD36 ligand, CD40 ligand, CD68 ligand, integrin β₁, β₂, β₃, or β₅ ligand, integrin αᵥβ₃ ligand, scavenger receptor ligand, phosphatidyl serine receptor ligand, β₂-glycoprotein I (β₂GP1) receptor ligand, scavenger receptor A (SR-A) ligand, macrophage receptor with collagenous structure (MARCO) ligand, scavenger receptor B1 (SR-B1) ligand, LOX-1 ligand, scavenger receptor that binds phosphatidylserine and oxidized lipoprotein (SR-PSOX) ligand, complement component C1q receptor ligand, complement component iC3b receptor ligand, lectin ligand, receptor activator of nuclear factor-κB ligand, CXCR1 ligand, CXCR2 ligand, CXCR3 ligand, CXCR4 ligand, CXCR5 ligand, CXCR6 ligand, CCR1 ligand, CCR2 ligand, CCR3 ligand, CCR4 ligand, CCR5 ligand, CCR6 ligand, CCR7 ligand, CCR8 ligand, CCR9 ligand, CX₃CR1 ligand, XCR1 ligand, PPARγ ligand, Galectin-3 ligand, molecule present at the surface apoptotic cells or secreted by them, and any other molecule that can modulate dendritic cell, monocyte, osteoclast, or macrophage gene expression and result in the modification of cellular phenotype, interferon-γ (IFN-γ), interleukin 1 (IL-1), IL-2, IL-3, IL-4 IL-5, IL-6, IL-7, IL-8, IL-9, IL-10, IL-11, IL-12, tumor necrosis factor α (TNF-α), transforming growth factor β (TGF-β), macrophage inflammatory protein 1α (MIP-1α), MIP-1β, MIP-2, MIP-3α, MIP-3β, SLC, I-309, TECK, fractalkine, lymphotactin, MCP-1α, MCP-1β, MCP-2, MCP-3, Eotaxin, MDC, TARC, phosphatidyl serine, GRO-α, ENA-78, NAP-2, IFN-γ-inducible protein 10 (IP-10), Mig, IFN-inducible T-cell alpha chemoattractant (I-TAC), stromal cell derived factor 1 (SDF-1), BCA-1, Bonzo, RANTES, ICAM-3, lysophosphatidyl choline, annexin I, β₂GP1, thrombospondin (TSP), oxidized low density lipoprotein (oxLDL), acetylated LDL, high density lipoprotein (HDL), advanced glycation endpoint LDL, milk fat globule protein (MFG), complement component iC3b, complement component C1q, granulocyte macrophage-colony stimulating factor (GM-CSF), macrophage-colony stimulating factor (M-CSF), apolipoprotein E (apoE), CD154, 12/15 lipoxygenase, Trance, and a fragment, derivative, or mimetic of such or any other molecule that can modulate dendritic cell, monocyte, osteoclast, or macrophage gene expression. Compositions comprising any one or more of the effectors are also provided.

All patents, patent applications, and other publications mentioned herein are incorporated by reference in their entirety. In the event of a conflict or inconsistency between any of the incorporated references and the instant specification or the understanding of one or ordinary skill in the art, the specification shall control. The determination of whether a conflict or inconsistency exists is within the discretion of the inventors and can be made at any time.

### DETAILED DESCRIPTION OF CERTAIN EMBODIMENTS OF THE INVENTION

For purposes of description, monocytes, macrophages, dendritic cells, osteoclasts, and cells of related cell types, are at times referred to herein as “macrophage-related cells”. In various embodiments of the invention related cell types include cells that differentiate from monocytes.

As used herein, a “phenotypic characteristic” can be any observable or detectable characteristic, property, attribute, or function of a cell. The phenotypic characteristic may be observed or detected in any of a number of ways. For example, a phenotypic characteristic may be observed or detected either by performing a test, observation, or measurement on the cell itself or by performing a test, observation, or measurement, on other cells, tissues, organs, etc., that may be affected by the cell, or by performing a test, observation, or measurement on a subject that contains the cell. The term “phenotype” includes any “phenotypic characteristic” and also refers more broadly to characteristics, properties, attributes, functions, etc., that may result from a combination of two or more phenotypic characteristics. Certain of these phenotypes may be defined with respect to an effect that macrophage-related cell(s) exhibiting the phenotype have on other cells or tissues either in vitro or in vivo (i.e., in a subject containing the macrophage-related cell(s), e.g., by performing a test, observation, or measurement, on other cells, tissues, organs, etc., that may be affected by the macrophage-related cell(s), or by performing a test, observation, or measurement on a subject that contains the macrophage-related cell(s).

Non-limiting examples of phenotypic characteristics that can be modulated (e.g., increased, decreased, temporally or spatially altered) by the present invention are: (i) expression of one or more genes (e.g., cytokines, inflammatory mediators, etc.); (ii) secretion of one or more molecules (e.g., cytokines, inflammatory mediators, etc.); (iii) migration to one or more sites in the body; (iv) ability to cause an alteration in one or more phenotypic characteristics or phenotypes of another macrophage-related cell or ability to cause an alteration in an alteration in one or more phenotypic characteristics or phenotypes of a non-macrophage-related cell, etc. A non-exhaustive list of the phenotypes that can be modified by an effector include pro-inflammatory, anti-inflammatory, immunogenic, tolerogenic, tissue destructive, tissue restorative, cytotoxic, migratory, bone-resorbing, pro-angiogenic, anti-angiogenic, suppressor, antigen presenting, or phagocytic. Additionally, many changes brought about by effector molecules cannot easily be assigned to one of these phenotypes but are still therapeutically relevant. Methods for observing, detecting, measuring, etc., these phenotypic characteristics and phenotypes are known in the art. For example, gene expression profiles can be assessed at the RNA level using cDNA or oligonucleotide microarray analysis, Northern blots, RT-PCR, etc. Protein expression can be measured using, for example, immunoblotting, immunohistochemistry, protein microarrays, etc. Various cell-based assays an and animal models can be used.

This invention leverages the intrinsic plasticity of dendritic cell, monocyte, osteoclast, and macrophage phenotypes to treat disease by changing at least one phenotypic characteristic or phenotype of dendritic cells, monocytes, osteoclasts, macrophages and/or other macrophage-related cells. The invention provides the recognition that the phenotype and function of such cells can be modulated or modified by any of a number of effector molecules for purposes of treating or preventing disease. In certain preferred embodiments of the invention the phenotypic characteristic is changed to a phenotypic characteristic that will slow or stop the progression of the disease. In other embodiments of the invention a phenotypic characteristic or phenotype is reduced or eliminated.

Certain macrophage phenotypes may be quantified in any of a variety of ways, e.g., the phenotype may be expressed in terms of a quantitative variable. In certain embodiments of the invention the quantitative variable is modified by at least 10%, 20%, 30%, 40%, 50%, 60%, 70%, 80%, 90%, or 100% relative to an initial value. In certain embodiments of the invention the phenotype is modified by at least 2, 3, 5, 10, 20, 50, or 100-fold relative to an initial value.

An effector can be, for example, a small molecule, a peptide, an oligopeptide, a polypeptide, a protein, an antibody, a synthetic binding molecule such as an affibody or other recombinantly produced binding molecule, an aptamer, an RNA molecule (e.g., a short interfering RNA, short hairpin RNA, antisense RNA, or ribozyme), a DNA molecule, an oligomer, a polymer, a lipid, a liposome, a cell (a prokaryotic or eukaryotic cell), or a virus. A non-exhaustive list of the classes of molecules may be used to modify the phenotype of dendritic cells, monocytes, osteoclasts, and macrophages in accordance with the present invention includes cytokines, chemokines, pattern recognition receptor ligands, hormones, adrenergic and cholinergic agonists, fatty acids, phospholipids, immunoglobulins or portions thereof, Fc domains of immunoglobulins, lipopolysaccharides (LPS), toll-like receptor (TLR) ligands, histamines, peroxisome proliferator-activated receptor ligands, CD14 ligands, CD36 ligands, CD40 ligands, CD68 ligands, β₂-glycoprotein I (β₂GP1) receptor ligands, integrin β₁, β₂, β₃, or β₅ ligands, integrin αᵥβ₃ ligands, complement component C1q receptor ligands, lectin ligands, receptor activator of nuclear factor-κB ligands, scavenger receptor ligands, SR-AI/SR-AII ligands, SR-B1 ligands, SR-PSOX ligands, macrophage receptor with collagenous structure (MARCO) ligands, LOX-1 ligands, CXCR1 ligands, CXCR2 ligands, CXCR3 ligands, CXCR4 ligands, CXCR5 ligands, CXCR6 ligands, CCR1 ligands, CCR2 ligands CCR3 ligands, CCR4 ligands, CCR5 ligands, CCR6 ligands CCR7 ligands, CCR8 ligands, CCR9 ligands, CX₃CR1 ligands, XCR1 ligands, PPARγ ligands, Galectin-3 ligands, molecules present at the surface apoptotic cells or secreted by them, and any other molecules that can modulate a phenotypic characteristic of a macrophage-related cell, e.g., expression of one or more genes, and preferably result in the modification of one or more phenotype(s).

Different effector molecules can have different effects on phenotypic characteristic(s) and/or phenotype(s) of dendritic cells, monocytes, osteoclasts, macrophages, and/or other macrophage-related cells. Some will modulate certain phenotypes, such as pro-inflammatory and cytotoxic, others will modulate different phenotypes, such as anti-inflammatory and phagocytic. With respect to any individual cell, some phenotypes are typically exclusive (i.e., an individual cell displays only one of the possible phenotypes simultaneously or within a given time window), such as anti-inflammatory and pro-inflammatory, while others are non-exclusive (i.e., an individual cell may display two or more of the phenotypes simultaneously or within a given time window), such as anti-inflammatory and phagocytic.

In certain embodiments phosphatidyl serine, M-CSF, TGF-β, IL-4, and IL-10 influence macrophage-related cells (e.g., macrophages) toward anti-inflammatory and phagocytic phenotypes and/or agents that antagonize or oppose M-CSF, TGF-β, IL-4, and/or IL-10 influence macrophage-related cells away from these phenotypes. In certain embodiments GM-CSF, IL-1, IL-2, and/or TNF-α influence the phenotype of macrophage-related cells (e.g., macrophages) toward pro-inflammatory and cytotoxic phenotypes and/or agents that antagonize or oppose GM-CSF, IL-1, IL-2, and/or TNF-α influence the phenotype of macrophage-related cells away from these phenotypes.

In accordance with the invention, a single effector or a combination of effectors is administered to a patient to modify at least one phenotypic characteristic or phenotype of dendritic cells, monocytes, osteoclasts, macrophages, and/or other macrophage-related cells. If multiple effectors are used, the effectors need not be administered together in a single composition, at the same time, via the same route of administration, although they may be. In some embodiments of this invention, effector(s) are administered systemically where they will modify the phenotype of dendritic cells, monocytes, osteoclasts, or macrophages present throughout the body. In other embodiments of this invention, effector(s) are administered locally, such as in the eye or at a site of a tumor.

In yet other embodiments of this invention, effector(s) are covalently or noncovalently attached to a targeting domain. The resulting conjugate may be considered to contain an effector domain comprising the effector, and a targeting domain. The targeting domain may, for example, result in the accumulation in a particular tissue or area, such as tumor, brain, maculas, lungs, joints, or sites of neovascularization. The targeting domain may, for example, be a ligand for a molecule expressed on a cell surface, a ligand for a molecule present in the extracellular matrix, etc. The targeting domain and the effector may be linked together by a permanent or a transient linkage. A transient linkage is defined as a linkage that has a half-time less than 1 month inside the body. A permanent linkage has a half-time of 1 month or greater inside the body.

In certain embodiments of the invention the effector is attached to two targeting domains, or consists of two targeting domains, at least one of which may act as an effector domain. A first targeting domain targets the effector to a target cell, e.g., a cell at a site of disease. The target cell may or may not be at least in part responsible for the disease or for a manifestation of the disease. A second targeting domain targets the effector to a macrophage, related cell, e.g., a macrophage. Thus a first targeting domain may be a ligand (target cell ligand) that binds to a target cell receptor.

The target cell receptor may, but need not be, specific to the target cell (e.g., it may be present on and/or expressed only by cells of the target cell type(s) or may be present on and/or expressed by one or more additional cell types). There may be target cells of multiple different cell types. In certain embodiments of the invention the target cell receptor is significantly present on and/or expressed by not more than a few cell types, e.g., between 1-5 different cell types, i.e., it is reasonably cell type specific. The second targeting domain may be a ligand (macrophage ligand) that binds to a macrophage receptor. The macrophage receptor may be present on and/or expressed only by macrophages or may be present on and/or expressed by one or more other cell types, e.g., other macrophage-related cells, immune system cells such as T and/or B lymphocytes, granulocytes, etc. Thus in certain embodiments of the invention the effector is bifunctional in that it includes first and second targeting domains that mediate binding to two different moieties, one of which mediates binding to a target cell and one of which mediates binding to a macrophage-related cell, e.g., a macrophage.

“Receptor” is used here in a broad sense to refer to any moiety that is present on or at the surface of a target cell so that it is accessible to binding by a targeting domain. A receptor may be a transmembrane or cell surface protein or portion thereof, or a carbohydrate modification of a transmembrane or cell surface protein. A receptor may, but need not be, recognized in the art as such. “Ligand” is also used here in a broad sense to refer to any moiety that can bind to a receptor. Ligands can be, e.g., small molecules (by which is meant organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol and have multiple carbon-carbon bonds.), peptides, polypeptides, nucleic acids, carbohydrates, lipids, etc. The ligand may be naturally occurring or synthetic. It may, but need not, be an endogenous ligand for the receptor to which it binds or may be a modified form thereof. A ligand can be part of a larger entity, wherein only the ligand mediates binding to the receptor. Binding of two or more moieties, e.g., a ligand and a receptor may be considered “specific” if the equilibrium dissociation constant, Kd is 10⁻³ M or less, preferably 10⁻⁴ M or less, more preferably 10⁻⁵ M or less, e.g., 10⁻⁶ M or less, 10⁻⁷ M or less, 10⁻⁸ M or less, or 10⁻⁹ M or less under the conditions of interest, e.g., under physiological conditions.

In the above embodiments there need not be a distinct effector domain that does not also act as a targeting domain. For example, in certain embodiments of the invention the effector modifies macrophage phenotype at least in part by directing the macrophage to a site of disease.

One of ordinary skill in the art will recognize that targeting and/or effector domains may be attached to one another in any of a variety of ways, either directly or via a rigid or flexible spacer moiety. For example, two polypeptide targeting domains may be synthesized as part of fusion protein. Small molecules can be attached to polypeptides via an amine group on a lysine residue. Suitable spacer or linker moieties can be, e.g., polypeptides such as (Gly)ₙ, where n typically ranges between 3 and 15, saturated or unsaturated hydrocarbon chains that may or may not be substituted at one or more positions and may include one or more aromatic or non-aromatic rings or heteroatoms either as part of the chain or as part of a substituent, etc.

Two or more components of a construct of the invention may be attached to one another by any of a number of methods that are well known in the art. In certain embodiments conjugation is accomplished using a linker. Standard linkers and linking methods include, but are not limited to, the glutaraldehyde method, which couples primarily through the α-amino group and ε-amino group, maleimide-sulfhydryl coupling chemistries (e.g., the maleimidobenzoyl-N-hydroxysuccinimide ester (MBS) method), and periodate oxidation methods. In addition, numerous cross-linking agents are known, which may be used to link two polypeptides or to link a polypeptide to a nonpolypeptide moiety. Suitable cross-linking agents include, e.g., carboiimides, N-Hydroxysuccinimidyl-4-azidosalicylic acid (NHS-ASA), dimethyl pimelimidate dihydrochloride (DMP), dimethylsuberimidate (DMS), 3,3′-dithiobispropionimidate (DTBP), etc.

For additional information on conjugation methods and crosslinkers see generally the journal Bioconjugate Chemistry, published by the American Chemical Society, Columbus Ohio, PO Box 3337, Columbus, Ohio, 43210. See also “Cross-Linking”, Pierce Chemical Technical Library, available at the Web site having URL www.piercenet.com and originally published in the 1994-95 Pierce Catalog and references cited therein and Wong S S, Chemistry of Protein Conjugation and Crosslinking, CRC Press Publishers, Boca Raton, 1991; and G. T. Hermanson, Bioconjugate Techniques, Academic Press, Inc., 1995.

Table 1 presents examples of a number of different target cell receptors, target cell ligands, macrophage receptors, and macrophage ligands. Each row of Table 1 lists a target cell receptor, target cell ligand, macrophage/immune receptor, and macrophage ligand. In accordance with the invention the target cell ligand and macrophage ligand are attached to one another, and the construct thus created can optionally include one or more effector domains such as any of those described herein. The construct is administered either locally or systemically to a subject. The target cell ligand binds to the target cell receptor, and the macrophage ligand binds to a macrophage-related cell. Table 1 also includes exemplary, non-limiting lists of diseases that could be treated using the corresponding target cell ligand/macrophage ligand construct. Table 1 also includes exemplary, non-limiting mechanisms of action of the target cell ligand/macrophage ligand construct.

Compstatin and derivatives thereof (also referred to as “analogs” are known in the art and are described, e.g., in WO2004/026328.

Coagulation factor VIIa (FVIIa) is a two chain, ˜50 kD vitamin K dependent serine protease that is generated by proteolysis of a catalytically inactive single chain precursor, FactorVII (FVII), which normally circulates at low levels in the blood. FVII undergoes a number of post-translational modifications including γ-carboxylation at ten residues and N-linked glycosylation at two positions prior to its secretion. Cleavage to produce the activated form occurs between residues 152 and 153 of the native human or bovine protein, resulting in two chains linked by a single disulfide bridge (Broze & Majerus, J. Biol. Chem., 255: 1242-1247, 1980). The activated protein binds with high affinity to exposed TF in the presence of calcium.

Factor VII and peptides derived therefrom (e.g., Factor VIIa-derived fragments) of use in the invention are described in WO90/03390 and U.S. Pat. No. 5,962,418. Alternately, an inactive form of Factor VIIa is used. In some embodiments of the invention inactive FVII or inactive FVIIa is a derivative of FVII or FVIIa that is catalytically inactivated in the active site, e.g., by derivatization with an inhibitor. Many irreversible serine protease inhibitors, which generally form covalent bonds with the protease active site, are known in the art. Examples of suitable inhibitors include peptide halomethyl ketones, e.g., peptide chloromethyl ketones (see, Williams et al., J. Biol. Chem. 264:7536-7540, 1989) or peptidyl halomethanes, e.g., peptidyl chloromethanes; azapeptides; acylating agents such as various guanidinobenzoate derivatives and the 3-alkoxy-4-chloroisocoumarins; sulphonyl fluorides such as phenylmethylsulphonylfluoride (PMSF); diisopropylfluorophosphate (DFP); tosylpropylchloromethyl ketone (TPCK); tosyllysylchloromethyl ketone (TLCK); nitrophenyl-sulphonates and related compounds; heterocyclic protease inhibitors such as isocoumarines, coumarins, organophosphor compound, and sulfonyl fluorides.

Examplary peptide chloromethyl ketones include Phe-Phe-Arg chloromethyl ketone, D-Phe-Phe-Arg chloromethyl ketone, L-Phe-Phe-Arg chloromethyl ketone, Phe-Pro-Arg chloromethyl ketone, D-Phe-Pro-Arg chloromethyl ketone, L-Phe-Pro-Arg chloromethyl ketone, Glu-Gly-Arg chloromethyl ketone, L-Glu-Gly-Arg chloromethyl ketone, D-Glu-Gly-Arg chloromethyl ketone, Dansyl-Phe-Phe-Arg chloromethyl ketone, Dansyl-D-Phe-Phe-Arg chloromethyl ketone, Dansyl-L-Phe-Phe-Arg chloromethyl ketone, Dansyl-Phe-Pro-Arg chloromethyl ketone, Dansyl-D-Phe-Pro-Arg chloromethyl ketone, Dansyl-L-Phe-Pro-Arg chloromethyl ketone, Dansyl-Glu-Gly-Arg chloromethyl ketone, Dansyl-L-Glu-Gly-Arg chloromethyl ketone, Dansyl-D-Glu-Gly-Arg chloromethyl ketone.

Examples of FVII or FVIIa inhibitors also include benzoxazinones or heterocyclic analogues thereof such as described in PCT/DK99/00138. Other inhibitors include, but are not limited to, small peptides, peptidomimetics; benzamidine systems; heterocyclic structures substituted with one or more amidino groups; aromatic or heteroaromatic systems substituted with one or more C(══NH)NHR groups in which R is H, C₁₋₃alkyl, OH or a group which is easily split off in vivo. It is noted that in addition to irreversible inhibitors, other classes of inhibitory agents can be used such as inhibitors which reversibly bind to FVII or FVIIa and are cleavable by FVIIa and inhibitors which reversibly bind to FVIIa but cannot be cleaved. For a review of inhibitors of serine proteases see Proteinase Inhibitors (Research Monographs in Cell and Tissue Physiology; v. 12) Elsevier Science Publishing Co., Inc., New York (1990).

Methods of using the above inhibitors to generate inactive FVII or FVIIa are known in the art. See, e.g., U.S. Pat. No. 5,817,788. A number of the above agents are commercially available and can be used in accordance with the instructions of the manufacturer. The inhibitor can be applied to FVII, which can then be cleaved to generate inactive FVIIa, or the inhibitor can be applied following cleavage of FVII to FVIIa.

Tissue factor is an ˜46 kD transmembrane glycoprotein that is a major initiator of the coagulation cascade (Nemerson, Y, Thromb Haemost., 74(1):180-4. 1995; Mackman N., Arterioscler Thromb Vasc Biol., 24(6):1015-22, 2004). It is composed of a hydrophilic extracellular domain, a membrane-spanning hydrophobic domain, and a short cytoplasmic tail. As noted above, TF is a receptor for plasma coagulation factor VIIa. In the presence of calcium, TF interacts with VIIa to form TF/VIIa complexes at the cell surface, which activate coagulation factors IX and X, ultimately leading to formation of an insoluble fibrin clot.

TF is normally expressed on the surface of a number of cell types that are not normally in contact with the blood but is not typically expressed or is expressed at a much lower level on endothelial cells in normal vasculature. However, TF is aberrantly expressed in a number of pathological states. In particular, TF is overexpressed on the surface of tumor cells of a variety of different types and on tumor-associated vascular endothelium (Contrino, J., et al., Nat. Med. 2:209-215, 1996; Shoji, M., et al., Am. J. Pathol. 152:399-411, 1998; Nishi, T., et al., Cancer 86:1354-1361, 1999; Koomagi, R. and Vohm, M., Int. J. Cancer 79:19-22, 1998).

Phosphatidylserine (PS) and PS analogs of use in the invention are described in U.S. Pub. No. 20050113297.

Other ligands and receptors listed in Table 1, e.g., complement component C3b, scavenger receptor A, G-rich oligonucleotides, etc., are known in the art.

One of skill in the art will recognize that any of the peptides or polypeptides described herein may be modified by addition, deletion, or substitution of one or more amino acids without substantially changing its activity. The modified form can be tested to determine whether it retains sufficient binding or other activity to be of use. Any such modified forms that retain sufficient activity are of use in the invention.

TABLE 1

| | Target cell | Target cell | Macrophage/ | | |
|---|---|---|---|---|---|
| Disease | receptor | ligand | Immune receptor | Mφ ligand | Mechanism |
| Neovasculature | Tissue | Factor VII | Phosphatidylserine | PS or PS | Phagocytic |
| in ocular | factor (TF) | or derived | (PS) receptor, milk | analog | clearance or |
| disease | | peptide | fat globule protein | | killing of |
| | | | E8 (MFG) | | neovasculature |
| Neovasculature | Griffonia | GSA | Phosphatidylserine | PS or PS | Phagocytic |
| in cancer | simplicifolia | antibody or | (PS) receptor, milk | analog | clearance or |
| | agglutinin | antibody | fat globule protein | | killing of |
| | (GSA) | domain | E8 (MFG) | | neovasculature |
| Cancer | Nucleolin | G-rich | Phosphatidylserine | PS or PS | Phagocytic |
| | | oligos | (PS) receptor, milk | analog | removal of cancer |
| | | (GROs) | fat globule protein | | cells |
| | | | E8 (MFG) | | |
| Cancer | ″ | G-rich | Scavenger receptor A | Carbohydrate | Phagocytic |
| | | oligos (GROs) | | ligands | removal of cancer |
| | | | | | cells (but may be |
| | | | | | proinflammatory) |
| Cancer | Colon | Anti-CEA | Phosphatidylserine | PS or analog | Phagocytic |
| | carcinoma | antibody or | (PS) receptor, milk | | removal of cancer |
| | embryonic | antibody | fat globule protein | | cells |
| | antigen | domain | E8 (MFG) | | |
| | (CEA) | | | | |
| Cancer | Colon | Anti-CEA | Scavenger receptor | Carbohydrate | Phagocytic |
| | carcinoma | antibody or | A (SR-A) | ligand | removal of cancer |
| | embryonic | antibody | | | cells but proinflammatory |
| | antigen | domain | | | |
| | (CEA) | | | | |
| Cancer | Nucleolin | G-rich | Scavenger receptor | Carbohydrate | Phagocytic |
| | | oligos | A (SR-A) | ligand | removal of cancer |
| | | (GROs) | | | cells |
| Arthritis, | Deposited | Compstatin | IL-10 or | IL-10 or TGF-β | Target |
| atherosclerosis. | C3b | and | TGF-β Receptor | | immunosuppressant |
| | | derivatives | | | to areas of |
| | | | | | complement |
| | | | | | deposition |
| Arthritis, | Deposited | Compstatin | Steroid receptors | Steroids | Concentrate |
| atherosclerosis. | C3b | and | (e.g., | | steroids at sites |
| | | derivatives | glucocorticoid) | | of C3b deposition |

Effectors may be administered as a pharmaceutical composition, which can contain any of a number of pharmaceutically acceptable adjuvants, carriers, excipients, etc., which are known in the art, a number of which are discussed elsewhere herein. See, e.g., U.S. Ser. No. 10/923,940, which is incorporated herein by reference. See, e.g., Remington's Pharmaceutical Sciences, 19th Ed., Easton, Pa., Mack Publishing Co., 1995. In certain embodiments the composition is a sustained or slow release composition.

A number of polymeric delivery vehicles for providing sustained or controlled release are available and can be used to administer the effectors of the invention. Various polymers, e.g., biocompatible polymers, which may be biodegradable, can be used. The active agent may be released as the polymer degrades. Polymers of use for drug delivery include, but are not limited to, poly(lactic-co-glycolic acid), polyanhydrides, ethylene vinyl acetate, polyglycolic acid, chitosan, polyorthoesters, polyethers, polylactic acid, and poly (beta amino esters). Peptides, proteins such as collagen and albumin, and dendrimers (e.g., PAMAM dendrimers, peptide dendrimers, etc.) may also be used.

In certain embodiments of the invention one or more additional pharmaceutical agents useful for treating or preventing the disease of interest, is also administered concurrently or in combination with one or more effectors of the invention.

A composition is generally administered in an amount and for a time sufficient to achieve a desired effect, e.g., a therapeutic effect such as reduction in severity or extent of at least one symptom or sign of a disease or condition; prevention of at least one symptom or sign of a disease or condition, etc.

The invention provides a composition, e.g., a pharmaceutical composition, containing any one or more of the effector agents mentioned above in an amount effective to modify the phenotype of a macrophage-related cell. The invention specifically provides a composition containing any two, any three, or any four of the afore-mentioned agents in an amount effective to modify at least one phenotypic characteristic of a macrophage-related cell.

The invention further provides a method of treating a subject comprising administering any of the inventive compositions to the subject in an amount effective to treat a disease, disorder, or condition that is at least in part directly or indirectly caused by or contributed to by macrophage-related cells or wherein macrophage-related cells play a role. The macrophage-related cells may not be directly responsible for the disease, disorder, or condition or a manifesation of the disease, disorder, or condition. The effective amount may be an amount sufficient to alleviate, inhibit, or reduce at least one symptom, sign, or manifestation of the disease, disorder, or condition. A treatment can be administered prophylactically, e.g., prior to development of a disease, disorder, or condition, or prior to a manifestation of a disease, disorder, or condition. A prophylactic treatment may reduce the likelihood that the subject will develop the disease, disorder, condition, or manifestation, or lessen its severity. A treatment can be administered after a disease, disorder, or condition, or a particular manifestation thereof, has developed. The treatment may inhibit further progression or worsening, result in an improvement and/or cure, etc.

Suitable preparations, e.g., substantially pure preparations of one or more effector agents of the invention may be combined with pharmaceutically acceptable carriers, diluents, solvents, etc., to produce an appropriate pharmaceutical composition. The invention therefore provides a variety of pharmaceutically acceptable compositions for administration to a subject comprising one or more effector agents of the invention and a pharmaceutically acceptable carrier, adjuvant, or vehicle.

Further provided are pharmaceutically acceptable compositions comprising a pharmaceutically acceptable derivative (e.g., a prodrug) of any of the effector agents of the invention, by which is meant any non-toxic salt, ester, salt of an ester or other derivative of a compound of this invention that, upon administration to a recipient, is capable of providing, either directly or indirectly, an effector agent of this invention or an active metabolite or residue thereof. As used herein, the term “active metabolite or residue thereof” means that a metabolite or residue thereof has at least 25% of the activity of the agent.

In various embodiments of the invention an effective amount of the pharmaceutical composition is administered to a subject by any suitable route of administration including, but not limited to, intravenous, intramuscular, by inhalation, by catheter, intraocularly, orally, rectally, intradermally, intrathecally, by application to the skin, etc.

Thus inventive compositions may be formulated for delivery by any available route including, but not limited to parenteral, oral, by inhalation to the lungs, nasal, bronchial, ophthalmic, transdermal (topical), transmucosal, rectal, and vaginal routes. The term “parenteral” as used herein includes subcutaneous, intravenous, intramuscular, intra-articular, intra-synovial, intrasternal, intrathecal, intrahepatic, intralesional and intracranial injection or infusion techniques. In certain embodiments a composition is administered intravenously. In certain embodiments a composition is administered intrathecally. In certain embodiments a composition is administered into a joint space.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Solvents, dispersion media, coatings, antibacterial and antifungal agents, isotonic and absorption delaying agents, and the like, compatible with pharmaceutical administration may be included. Supplementary active agents, e.g., agents independently active against the disease or clinical condition to be treated, or agents that enhance activity of a compound, can also be incorporated into the compositions.

Pharmaceutically acceptable salts include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium and potassium), alkaline earth metal (e.g., magnesium), ammonium and N+(C1-4 alkyl)4 salts. This invention also envisions the quaternization of any basic nitrogen-containing groups of the compounds disclosed herein. Water or oil-soluble or dispersible products may be obtained by such quaternization.

A pharmaceutical composition is formulated to be compatible with its intended route of administration. Solutions or suspensions used for parenteral (e.g., intravenous), intramuscular, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use typically include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.), phosphate buffered saline (PBS), or Ringer's solution.

Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Preferred pharmaceutical formulations are sterile, if possible, and are stable under the conditions of manufacture and storage. They may preserved against the contaminating action of microorganisms such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of injectable compositions can be brought about by including in the composition an agent which delays absorption, for example, aluminum monostearate and gelatin. Prolonged absorption of oral compositions can be achieved by various means including encapsulation.

Sterile injectable solutions can be prepared by incorporating the active compound in the required amount in an appropriate solvent with one or a combination of ingredients enumerated above, as required, followed by filtered sterilization. Preferably solutions for injection are free of endotoxin. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Formulations for oral delivery may advantageously incorporate agents to improve stability within the gastrointestinal tract and/or to enhance absorption.

For administration by inhalation, the effectors are preferably delivered in the form of an aerosol spray from a pressured container or dispenser which contains a suitable propellant, e.g., a gas such as carbon dioxide, or a nebulizer. Liquid or dry aerosol (e.g., dry powders, large porous particles, etc.) can be used. The present invention also contemplates delivery of compositions using a nasal spray.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2_octyldodecanol, benzyl alcohol and water.

For local delivery to the eye, the pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum. Preferred methods of local administration to the eye include, e.g., choroidal injection, transscleral injection or placing a scleral patch, selective arterial catheterization, intraocular administration including transretinal, subconjunctival bulbar, intravitreous injection, suprachoroidal injection, subtenon injection, scleral pocket and scleral cutdown injection, by osmotic pump, etc.

It is typically advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

A therapeutically effective amount of a pharmaceutical composition typically ranges from about 0.001 to 100 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The pharmaceutical composition can be administered at various intervals and over different periods of time as required, e.g., multiple times per day, daily, every other day, once a week for between about 1 to 10 weeks, between 2 to 8 weeks, between about 3 to 7 weeks, about 4, 5, or 6 weeks, etc. The skilled artisan will appreciate that certain factors can influence the dosage and timing required to effectively treat a subject, including but not limited to the severity of the disease or disorder, previous treatments, the general health and/or age of the subject, and other diseases present. Generally, treatment of a subject with an inventive composition can include a single treatment or, in many cases, can include a series of treatments.

Exemplary doses include milligram or microgram amounts of the inventive compounds per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram.) For local administration (e.g., intranasal), doses much smaller than these may be used. It is furthermore understood that appropriate doses depend upon the potency of the agent, and may optionally be tailored to the particular recipient, for example, through administration of increasing doses until a preselected desired response is achieved. It is understood that the specific dose level for any particular subject may depend upon a variety of factors including the activity of the specific compound employed, the age, body weight, general health, gender, and diet of the subject, the time of administration, the route of administration, the rate of excretion, any drug combination, the severity of the disease, disorder, or condition, etc.

The invention also encompasses gene therapy, in which a nucleic acid vector that encodes a therapeutic effector agent of this invention, e.g., a therapeutic nucleic acid such as an shRNA) or a therapeutic polypeptide, in operable association with regulatory elements sufficient to direct expression of the operably linked nucleic acid, is introduced into a subject. Nucleic acids can be introduced into a subject by any of a number of methods. For instance, a pharmaceutical preparation of a nucleic acid vector can be introduced systemically, e.g., by intravenous injection. Expression of the polypeptide in particular target cells may result from specificity of transfection provided by the vector, cell-type or tissue-type expression due to the transcriptional regulatory sequences controlling expression of the gene, or a combination thereof. Alternatively, initial delivery of the nucleic acid can be more limited. For example, a genetic vector can be locally administered.

A pharmaceutical composition can comprise a nucleic acid or a genetic vector in an acceptable diluent or carrier, or can comprise a slow release matrix in which the nucleic acid or genetic vector is encapsulated, entrapped, or embedded. The genetic vector can be a plasmid, virus, or other vector. Alternatively, the pharmaceutical composition can comprise one or more cells which produce a therapeutic nucleic acid or polypeptide. Preferably such cells secrete the nucleic acid or polypeptide into the extracellular space or bloodstream.

Viral vectors that are of use include, but are not limited to, retroviruses, lentiviruses, other RNA viruses such as poliovirus or Sindbis virus, adenovirus, adeno-associated virus, herpes viruses, SV 40, vaccinia and other DNA viruses. Replication-defective murine retroviral or lentiviral vectors are widely utilized gene transfer vectors. Chemical methods of gene delivery can involve carrier-mediated gene transfer through the use of fusogenic lipid vesicles such as liposomes or other vesicles for membrane fusion. A carrier harboring a nucleic acid of interest can be introduced into the vascular system or other body fluids or administered locally. The carrier can be site specifically directed to a target organ or tissue in the body. Cell or organ-specific DNA-carrying liposomes, for example, can be developed and the foreign nucleic acid carried by the liposome becomes attached to or taken up by those specific cells. Carrier mediated gene transfer may also involve the use of lipid-based compounds which are not liposomes. For example, lipofectins and cytofectins are lipid-based compounds containing positive ions that bind to negatively charged nucleic acids and form a complex that can ferry the nucleic acid across a cell membrane. Cationic polymers are known to spontaneously bind to and condense nucleic acids such as DNA into nanoparticles. For example, naturally occurring proteins, peptides, or derivatives thereof have been used. Synthetic cationic polymers such as polyethylenimine (PEI), polylysine (PLL) etc., are also known to condense DNA and are useful delivery vehicles. Dendrimers can also be used.

Many of the useful polymers contain both chargeable amino groups, to allow for ionic interaction with the negatively charged DNA phosphate, and a degradable region, such as a hydrolyzable ester linkage. Examples of these include poly(alpha-(4-aminobutyl)-L-glycolic acid), network poly(amino ester), and poly(beta-amino esters). These complexation agents can protect DNA against degradation, e.g., by nucleases, serum components, etc., and create a less negative surface charge, which may facilitate passage through hydrophobic membranes (e.g., cytoplasmic, lysosomal, endosomal, nuclear) of the cell. Certain complexation agents facilitate intracellular trafficking events such as endosomal escape, cytoplasmic transport, and nuclear entry, and can dissociate from the nucleic acid.

The following sections describe some of the diseases that may be treated and/or prevented by the present invention and provides further details regarding the methods of the invention.

Atherosclerosis

Atherosclerosis, which can be considered to be a pathological remodeling of the arteries, is a major cause of morbidity and mortality in developed countries and is the underlying basis of myocardial infarction, stroke and peripheral artery disease. Atherosclerosis can be considered as an unusual form of chronic inflammation occurring within the artery wall. Fatty streaks, the earliest detectable lesions in atherosclerosis, contain macrophage-derived foam cells whose phenotype is different from recruited blood monocytes. Monocytes are recruited to tissues via constitutive signals and in response to inflammatory mediators.

More advanced atherosclerotic lesions, called fibro-fatty plaques, are the result of continued monocyte recruitment, together with smooth muscle cell migration and proliferation, and can contain CD4+ T cells. Chemokines or chemoattractant cytokines constitute a family of over 40 different cell signaling molecules important for constitutive trafficking and recruitment of leukocytes in response to inflammatory mediators. Some chemokines that can act as potent mediators of monocyte migration and macrophage differentiation are expressed in human atherosclerotic lesions. Indeed, as discussed below, experiments performed with gene-knockout mice lacking macrophage chemoattractant protein 1 (MCP-1) have suggested an important role for the CC chemokine MCP-1 and its specific receptor CCR2 in the initial stages of atherogenesis. In recent years, pathologists have advanced the idea of stable and unstable (or vulnerable) atherosclerotic plaques. Stable plaques are characterized by a thick fibrous cap overlying a plaque that does not contain a cholesterol-rich necrotic core. By contrast, unstable plaques have a thin fibrous cap, contain a higher ratio of macrophages to smooth muscle cells, and have a lipid-filled necrotic core. Unstable plaques are more likely to rupture, which exposes the thrombogenic core of the lesion to arterial blood. This leads to platelet aggregation and the formation of an arterial thrombus attached to the vessel wall. Thrombus material can break away from the wall and be transported to a distant site (embolism), where it may lead to blockage of smaller arteries. The clinical consequences of arterial thrombosis are heart attacks, strokes and renal disease. Indeed, the majority (˜60%) of arterial thrombosis is associated with ruptured atherosclerotic plaques. Another commonly observed feature of atherosclerotic plaques is endothelial cell denudation (plaque erosion) and other changes in the endothelial cells that predispose to arterial thrombosis and its clinical sequelae.

Macrophages are intrinsically involved with atherosclerosis. Macrophages are at least partially responsible for tissue remodeling. For example, they secrete many different cytokines, growth factors and proteases that facilitate the remodeling of the extracellular matrix and encourage the recruitment of other cell types such as fibroblasts and smooth muscle cells. Additionally, macrophages and macrophage-derived foam cells are a major constituent of atherosclerotic plaques, a higher ratio of macrophages being associated with unstable plaques.

The invention provides a method of treating or preventing atherosclerosis comprising administering an effector that alters at least one phenotypic characteristic of a macrophage or macrophage-related cell. The alteration Administering effectors that change the phenotype of macrophages and makes them less likely to cause and participate in atherosclerosis plaques by modifying the cytokines, growth factors, and proteases they secrete and by preventing them from becoming a constituent of these plaques will form the base of a very effective treatment against atherosclerosis.

Multiple Sclerosis

Multiple sclerosis (MS) usually begins in early adulthood with an autoimmune inflammatory “strike” against components of the myelin sheath. Paralysis, sensory disturbances, lack of coordination and visual impairment are common features. The disease often starts with an “attack” that lasts from a few days to weeks; this is followed by remission that lasts from a few months to years. This relapsing-remitting phase often lasts five to ten years, but approximately 30% of individuals with this form of MS enter a secondary chronic-progressive state. This chronic-progressive state is often characterized by the inability to walk, which leaves the MS patient wheelchair-bound. In the chronic-progressive phase, distinct attacks are rare and the disease progresses insidiously. Occasionally, however, clinical disability begins with this progressive phase, in which case the disease is called “primary-progressive MS”. Evidence indicates that the earlier phase of disease, characterized by distinct attacks followed by remission, may be mediated by an autoimmune reaction. The subsequent chronic phase of disease is due to degeneration of both the myelin sheath, which is synthesized by oligodendroglial cells, and the underlying axon, which emanates from the neuronal cell body some distance away. Indeed it is axon loss in the spinal cord and spinal cord atrophy that correlate most strongly with the inability to walk and paralysis.

Worldwide, approximately 1,000,000 individuals are afflicted with MS. Women with the disease outnumber men two to one. This bias towards females is seen in other autoimmune diseases, for example, rheumatoid arthritis, systemic lupus erythematosus and thyroiditis. Genome-wide studies have revealed that susceptibility to MS is linked to genes in the major histocompatibility complex (MHC) on chromosome 6. Alleles for certain class II genes, HLA-DR and HLA-DQ, confer the strongest risk of contracting MS. Other genes within the HLA complex may be involved in the pathogenesis of MS, including expression of tumor necrosis factor-α (TNF-α), various components of the complement cascade and myelin oligodendroglial glycoprotein. More recently, transcriptional profiling with gene microarrays and large-scale sequencing of transcripts from MS lesions have identified a number of genes that are involved in the pathogenesis of acute disease. These include immunoglobulin and interleukin 6 (IL-6) as well as osteopontin, which plays a role in the transition from relapsing-remitting to chronic MS.

Hence various proteins are used, in a modular manner, to build structures whose intrinsic components can resemble each other. If a human and a foreign entity such as an infectious agent (e.g., virus, bacteria, fungus, protozoa, etc.) invading that human share a common epitope, or a gene sequence that encodes one of these conserved structural motifs, the immune system, in recognizing a structure on this entity, may mistakenly also attack “self”. In the context of MS, many protein sequences expressed by infectious agents share homologies with structures found on the myelin sheath; this leads to an attack on myelin via a process called molecular mimicry. Relapses in MS are often triggered by common viral infections. Viruses such as herpesvirus, influenza, measles, papilloma virus and Epstein-Barr Virus all have genes encoding sequences that mimic those found in the major structural proteins of myelin. Indeed, antibodies to components of the myelin sheath cross-react and bind sequences from these microbes. T cells also recognize sequences from the myelin sheath that are shared with these microbial sequences6. Once a T cell, B cell or macrophage is activated by a foreign microbe, self-protein or microbial superantigen, it may penetrate the blood-brain barrier.

Penetration of the blood-brain barrier by activated lymphocytes is a multistep process. There are specialized capillary endothelial cells in the central nervous system (CNS) that are nonfenestrated and connected through tight junctions. During the inflammatory response, TNF-α and interferon-β (IFN-β) induce these capillary endothelial cells to express vascular cell adhesion molecule (VCAM) and MHC class II molecules. Activated T cells express integrins, such as very late antigen (VLA-4), and members of the immunoglobulin superfamily, such as CD4, that can bind VCAM and MHC class II molecules, respectively. Once activated, any T cell expressing VLA-4, for example, can bind to adhesion molecules on the surface of inflamed endothelium and “walk-through” the endothelium. In an animal model of MS, acute experimental autoimmune encephalomyelitis (EAE), blockade of VLA-4 reverses clinical paralysis and prevents further relapses in the chronic model of this disease. In acute MS lesions, VLA-4 is found on T cells that collect in the “perivascular lymphocyte cuff”, a region around veins and capillaries that is limited by the extracellular matrix. Clinical studies with a human antibody to VLA-4 are now in Phase III following promising Phase II trial results in which the incidence of MS relapses was reduced.

Once the activated lymphocytes have extravasated, they still must pass through a barrier of extracellular matrix, comprised of type IV collagen, before they can enter the CNS. Matrix metalloproteases (MMPs) are a family of structurally and functionally related enzymes that are involved in the degradation of the extracellular matrix as well as the proteolysis of myelin components in MS. MMPs contain Zn²⁺ at their active site, show TNF-α convertase activity and induce the cleavage of TNF-α from a cell-bound to a soluble form. Gelatinase A and B (also called MMP2 and MMP9) play a key role in penetration of the extracellular matrix. These MMPs are detectable in the spinal fluid of MS patients, and gelatinase B immunoreactivity is present in endothelial cells, pericytes, macrophages and astrocytes of MS lesions. Myelin-specific T cell clones derived from MS patients also produce gelatinase B upon activation with antigen. The presence of gelatinase B in the perivascular infiltrate is associated with disruption of the type IV collagen-positive basement membrane and is critical in the opening of the blood-brain barrier. Once the blood-brain barrier is breached, inflammatory cells spread into the white matter of the CNS. MMP inhibition by tissue inhibitors of matrix metalloproteases (TIMPs) can block TNF-α and thereby down-regulate the induction of adhesion molecules such as VCAM. TIMP-1 is present in the spinal fluid of MS patients and is inducible by various cytokines, including TNF-α. In terms of MS therapy, IFN-β, a potent inhibitor of gelatinase B activity, has been used relatively successfully in clinical trials. Inhibition of gelatinase may interfere with T cell migration into the CNS as well as T cell secretion of TNF-α. Other MMP inhibitors are currently under intense development for MS.

Once immune cells have spread to the white matter of the CNS, the immune response is targeted to the entire supramolecular myelin complex. Antibodies to various myelin proteins and lipids of the myelin sheath, as well as to molecules expressed in the CNS, are secreted by B cells that have migrated to the brain or from serum that has extravasated across the blood-brain barrier. Activated complement proteins appear in the spinal fluid along with membrane-attack complexes, which represent the terminal components of this cascade. T cells target certain proteins normally found in the myelin sheath. These include myelin basic protein, myelin oligodendroglial glycoprotein and proteolipid protein, as well as stress proteins such as B crystallin, which is found in the myelin sheath after activation via the inflammatory response. The T cells produce cytokines, notably lymphotoxin-α (LT-α) and TNF-α, which are members of the TNF family. LT-α is secreted as a LT-3a homotrimer and, like TNF-α, can bind to the p55 TNF receptor (p55-TNFRI) or the p75 TNFR (p75-TNFRII). These cytokines induce macrophages, microglial cells and astrocytes to produce NO and osteopontin.

The free radical NO is a major mediator in autoimmune diseases. NO is involved in the killing of oligodendroglial cells by microglia. Nitric oxide synthase (iNOS), which catalyzes NO synthesis, has been found in demyelinating lesions in MS. Both IFN-β and TNF-α induce iNOS transcription in astrocytes, microglia and macrophages. The combined effect of antibody, complement, NO and TNF-α damages myelin and induces the macrophage to phagocytose large chunks of the myelin sheath. In addition, macrophages and T cells produce osteopontin. This induces more T helper subset 1 (TH1) cytokines, including IFN-β and IL-12, and down-regulates TH2 cytokines such as IL-10. TH1 cytokines may exacerbate MS, whereas TH2 cytokines may reduce the extent of MS lesions. This concerted attack by T cells, B cells, complement and inflammatory mediators such as cytokines, osteopontin and NO produces areas of demyelination, which impairs electrical conduction along the axon and produces the pathophysiological defect.

Macrophages are involved at the core of the pathogenesis of MS, by secreting MMP-9, NO, TNF-α, IL-12, IFN-β, osteopontin, and other factors that enable the disease to progress. In an embodiment of the invention modifying the phenotype of macrophages to become anti-inflammatory and reduce the expression level of MMP-9, NO, TNF-α, IL-12, IFN-β, and/or osteopontin inhibits or prevents the progress of MS and reduces its debilitating effects.

Equivalents and Scope

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. In the claims articles such as “a,”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. In particular, any claim that is dependent on another claim can be modified to include one or more limitations found in any other claim that is dependent on the same base claim. Furthermore, where the claims recite a composition, it is to be understood that methods of administering the composition according to any of the methods disclosed herein, methods of using the composition for any of the purposes disclosed herein are included, and methods of making the composition according to any of the methods of making disclosed herein are included, unless otherwise indicated or unless it would be evident to one of ordinary skill in the art that a contradiction or inconsistency would arise. The invention includes embodiments that encompass every possible permutation of (i) an effector agent or agent(s), (ii) a phenotype or phentype(s), (iii) macrophage-related cell type or types, and (iv) a disease or condition to be treated. For example, in a representative embodiment, the effector agent is MIP-1α, the phenotype is inflammatory, the cell type is a macrophage, and the disease or condition is atherosclerosis. In another representative embodiment, the effector agent is ICAM-3, the phenotype is bone-resorbing, the cell type is an osteoclast, and the disease or condition is osteoporosis. Further provided are every possible permutation of delivery vehicle, administration route, targeting moiety, and general class of effector agent (e.g., small molecule, antibody, siRNA, etc.), in combination with a phenotype or phentype(s), a macrophage-related cell type or types, and a disease or condition to be treated.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein.

Wherever the claims recite “a method of modifying a phenotypic characteristic or phenotype of a macrophage-related cell in the body of a subject or comprising administering an effector to the subject . . . ”, the invention also includes a method of treating a subject by administering the effector to the subject in the same way and/or including the same claim elements or substantially similar claim elements. Wherever the claims recite “a method of modifying a phenotypic characteristic or phenotype of a macrophage-related cell in the body of a subject or comprising administering an effector to the subject . . . ”, the invention also includes a method of modifying a phenotypic characteristic or phenotype of a macrophage-related cell by contacting the cell with the effector in vitro (i.e., outside the body of a subject), wherein the claim includes the same or substantially similar claim elements, unless such claim element(s) relate specifically to administration to a subject. Any of the embodiments of the invention that include administering a composition to a subject can include a step of providing a subject, e.g., a subject at risk of or suffering from a disease, disorder, or condtion. The methods may include a step of diagnosing a subject as suffering from or at risk of a disease, disorder, or condition.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any one or more embodiments, variations, elements, diseases, conditions, effectors, administration routes, phenotypes, cell types, etc., may be explicitly excluded from any one or more of the claims. For purposes of brevity, these various embodiments in which one or more elements, diseases, conditions, effectors, administration routes, phenotypes, cell types, etc., is excluded from the claims are not set forth individually herein but are included in the invention.

What is claimed is:

1. A method of modifying a phenotypic characteristic or phenotype of a macrophage-related cell in the body of a subject or comprising administering an effector to the subject, wherein the effector is administered in an amount sufficient to cause a change in at least one phenotypic characteristic or phenotype of the macrophage-related cell, and wherein the macrophage-related cell is selected from the group consisting of: dendritic cells, monocytes, osteoclasts, and macrophages, and wherein administration of the effector optionally results in a change in the expression level of at least one gene in the macrophage-related cell.

2. The method of claim 1, wherein the administering step comprises administering a composition containing one or more effectors and, optionally, one or more adjuvants, carriers, or excipients.

3. The method of claims 1, wherein the phenotypic characteristic or phenotype is modulated only in macrophages, monocytes, dendritic cells, or osteoclasts, or any combination of three or fewer of these cell types.

4. The method of claim 1, wherein only the phenotypic characteristic or phenotype of macrophage-related cells present in a particular region or tissue is modulated.

5. The method of claim 1, wherein the effector is selected from the group consisting of: a cytokine, chemokine, pattern recognition receptor ligand, hormone, adrenergic and cholinergic agonist, fatty acid, phospholipid, immunoglobulin, Fc domain of immunoglobulins, lipopolysaccharide (LPS), toll-like receptor (TLR) ligand, histamine, peroxisome proliferator-activated receptor ligand, CD14 ligand, CD36 ligand, CD40 ligand, CD68 ligand, integrin β₁, β₂, β₃, or β₅ ligand, integrin αᵥβ₃ ligand, scavenger receptor ligand, phosphatidyl serine receptor ligand, β₂-glycoprotein I (β₂GP1) receptor ligand, scavenger receptor A (SR-A) ligand, macrophage receptor with collagenous structure (MARCO) ligand, scavenger receptor B1 (SR-B1) ligand, LOX-1 ligand, scavenger receptor that binds phosphatidylserine and oxidized lipoprotein (SR-PSOX) ligand, complement component C1q receptor ligand, complement component iC3b receptor ligand, lectin ligand, receptor activator of nuclear factor-κB ligand, CXCR1 ligand, CXCR2 ligand, CXCR3 ligand, CXCR4 ligand, CXCR5 ligand, CXCR6 ligand, CCR1 ligand, CCR2 ligand, CCR3 ligand, CCR4 ligand, CCR5 ligand, CCR6 ligand, CCR7 ligand, CCR8 ligand, CCR9 ligand, CX₃CR1 ligand, XCR1 ligand, PPARγ ligand, Galectin-3 ligand, molecule present at the surface apoptotic cells or secreted by them, and any other molecule that can modulate dendritic cell, monocyte, osteoclast, or macrophage gene expression and result in the modification of cellular phenotype.

6. The method of claim 1, wherein the effector is selected from the group consisting of interferon-γ (IFN-γ), interleukin 1 (IL-1), IL-2, IL-3, IL-4 IL-5, IL-6, IL-7, IL-8, IL-9, IL-10, IL-11, IL-12, tumor necrosis factor α (TNF-α), transforming growth factor β (TGF-β), macrophage inflammatory protein 1α (MIP-1α), MIP-1β, MIP-2, MIP-3α, MIP-3β, SLC, I-309, TECK, fractalkine, lymphotactin, MCP-1α, MCP-1β, MCP-2, MCP-3, Eotaxin, MDC, TARC, phosphatidyl serine, GRO-α, ENA-78, NAP-2, IFN-γ-inducible protein 10 (IP-10), Mig, IFN-inducible T-cell alpha chemoattractant (I-TAC), stromal cell derived factor 1 (SDF-1), BCA-1, Bonzo, RANTES, ICAM-3, lysophosphatidyl choline, annexin I, β₂GP1, thrombospondin (TSP), oxidized low density lipoprotein (oxLDL), acetylated LDL, high density lipoprotein (HDL), advanced glycation endpoint LDL, milk fat globule protein (MFG), complement component iC3b, complement component C1q, granulocyte macrophage-colony stimulating factor (GM-CSF), macrophage-colony stimulating factor (M-CSF), apolipoprotein E (apoE), CD154, 12/15 lipoxygenase, Trance, and a fragment, derivative, or mimetic of such or any other molecule that can modulate dendritic cell, monocyte, osteoclast, or macrophage gene expression.

7. The method of claim 1, wherein the effector modifies the expression level of a factor selected from the group consisting of CD11b, CD14, CD68, FcγR, MHC-II, esterase, osteopontin, IFN-γ, TNF-γ, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, MIP-1, MIP-2, MIP-3, MCP-1, MCP-2, MCP-3, brain-derived neurotrophic factor, TRAP, calcitonin receptor, tissue plasminogen activator (tPA), urokinase plasminogen activator (uPA), matrix metalloprotease 1 (MMP-1), MMP-2, MMP-3, MMP-7, MMP-8, MMP-9, MMP-10, MMP-11, MMP-12, MMP-13, MMP-14, MMP-15, MMP-16, MMP-17, MMP-18, MMP-19, MMP-20, MMP-21, MMP-22, MMP-23, MMP-24, MMP-25, MMP-26, MMP-27, MMP-28, SR-A, MARCO, 12/15 lipoxygenase, CD36, SR-B1, CD68, LOX-1, SR-PSOX, Galectin-3, fibroblast growth factors (FGFs), vascular endothelial growth factors (VEGF), platelet-derived growth factor (PDGF), and cathepsin K.

8. The method of claim 1, wherein the effector is selected from the group consisting of an antibody, an aptamer, or a ligand that binds one of more effectors selected from the group consisting of: a cytokine, chemokine, pattern recognition receptor ligand, hormone, adrenergic and cholinergic agonist, fatty acid, phospholipid, immunoglobulin, Fc domain of immunoglobulins, lipopolysaccharide (LPS), toll-like receptor (TLR) ligand, histamine, peroxisome proliferator-activated receptor ligand, CD14 ligand, CD36 ligand, CD40 ligand, CD68 ligand, integrin β₁, β₂, β₃, or β₅ ligand, integrin αᵥβ₃ ligand, scavenger receptor ligand, phosphatidyl serine receptor ligand, β₂-glycoprotein I (β₂GP1) receptor ligand, scavenger receptor A (SR-A) ligand, macrophage receptor with collagenous structure (MARCO) ligand, scavenger receptor B1 (SR-B1) ligand, LOX-1 ligand, scavenger receptor that binds phosphatidylserine and oxidized lipoprotein (SR-PSOX) ligand, complement component C1q receptor ligand, complement component iC3b receptor ligand, lectin ligand, receptor activator of nuclear factor-κB ligand, CXCR1 ligand, CXCR2 ligand, CXCR3 ligand, CXCR4 ligand, CXCR5 ligand, CXCR6 ligand, CCR1 ligand, CCR2 ligand, CCR3 ligand, CCR4 ligand, CCR5 ligand, CCR6 ligand, CCR7 ligand, CCR8 ligand, CCR9 ligand, CX₃CR1 ligand, XCR1 ligand, PPARγ ligand, Galectin-3 ligand, molecule present at the surface apoptotic cells or secreted by them, any other molecule that can modulate dendritic cell, monocyte, osteoclast, or macrophage gene expression and result in the modification of cellular phenotype, interferon-γ (IFN-γ), interleukin 1 (IL-1), IL-2, IL-3, IL-4 IL-5, IL-6, IL-7, IL-8, IL-9, IL-10, IL-11, IL-12, IL-13, IL-14, IL-15, IL-16, IL-17, IL-18, IL-19, IL-20, IL-21, tumor necrosis factor α (TNF-α), transforming growth factor β (TGF-β), macrophage inflammatory protein 1α (MIP-1α), MIP-1β, MIP-2, MIP-3α, MIP-3β, SLC, I-309, TECK, fractalkine, lymphotactin, MCP-1α, MCP-1β, MCP-2, MCP-3, Eotaxin, MDC, TARC, phosphatidyl serine, GRO-α, ENA-78, NAP-2, IFN-γ-inducible protein 10 (IP-10), Mig, IFN-inducible T-cell alpha chemoattractant (I-TAC), stromal cell derived factor 1 (SDF-1), BCA-1, Bonzo, RANTES, ICAM-3, lysophosphatidyl choline, annexin I, β₂GP1, thrombospondin (TSP), oxidized low density lipoprotein (oxLDL), acetylated LDL, high density lipoprotein (HDL), advanced glycation endpoint LDL, milk fat globule protein (MFG), complement component iC3b, complement component C1q, granulocyte macrophage-colony stimulating factor (GM-CSF), macrophage-colony stimulating factor (M-CSF), apolipoprotein E (apoE), CD154, 12/15 lipoxygenase, Trance, and a fragment, derivative, or mimetic of such or any other molecule that can modulate dendritic cell, monocyte, osteoclast, or macrophage gene expression and a receptor for a ligand that binds one of the effectors selected from the group consisting of interferon-γ (IFN-γ), interleukin 1 (IL-1), IL-2, IL-3, IL-4 IL-5, IL-6, IL-7, IL-8, IL-9, IL-10, IL-11, IL-12, tumor necrosis factor α (TNF-α), transforming growth factor β (TGF-β), macrophage inflammatory protein 1α (MIP-1α), MIP-1β, MIP-2, MIP-3α, MIP-3β, SLC, I-309, TECK, fractalkine, lymphotactin, MCP-1α, MCP-1β, MCP-2, MCP-3, Eotaxin, MDC, TARC, phosphatidyl serine, GRO-α, ENA-78, NAP-2, IFN-γ-inducible protein 10 (IP-10), Mig, IFN-inducible T-cell alpha chemoattractant (I-TAC), stromal cell derived factor 1 (SDF-1), BCA-1, Bonzo, RANTES, ICAM-3, lysophosphatidyl choline, annexin I, β₂GP1, thrombospondin (TSP), oxidized low density lipoprotein (oxLDL), acetylated LDL, high density lipoprotein (HDL), advanced glycation endpoint LDL, milk fat globule protein (MFG), complement component iC3b, complement component C1q, granulocyte macrophage-colony stimulating factor (GM-CSF), macrophage-colony stimulating factor (M-CSF), apolipoprotein E (apoE), CD154, 12/15 lipoxygenase, Trance, and a fragment, derivative, or mimetic of such or any other molecule that can modulate dendritic cell, monocyte, osteoclast, or macrophage gene expression. CD11b, CD14, CD68, FcγR, MHC-II, esterase, ostepontin, IFN-γ, TNF-α, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, MIP-1, MIP-2, MIP-3, MCP-1, MCP-2, MCP-3, brain-derived neurotrophic factor, TRAP, calcitonin receptor, tissue plasminogen activator (tPA), urokinase plasminogen activator (uPA), matrix metalloprotease 1 (MMP-1), MMP-2, MMP-3, MMP-7, MMP-8, MMP-9, MMP-10, MMP-11, MMP-12, MMP-13, MMP-14, MMP-15, MMP-16, MMP-17, MMP-18, MMP-19, MMP-20, MMP-21, MMP-22, MMP-23, MMP-24, MMP-25, MMP-26, MMP-27, MMP-28, SR-A, MARCO, 12/15 lipoxygenase, CD36, SR-B1, CD68, LOX-1, SR-PSOX, Galectin-3, fibroblast growth factors (FGFs), vascular endothelial growth factors (VEGF), platelet-derived growth factor (PDGF), and cathepsin K.

9. The method of claim 1, wherein the effector contains one or more targeting domains and one of more effector domains, where the effector domain(s) is a derivative of an effector selected from the group consisting of a cytokine, chemokine, pattern recognition receptor ligand, hormone, adrenergic and cholinergic agonist, fatty acid, phospholipid, immunoglobulin, Fc domain of immunoglobulins, lipopolysaccharide (LPS), toll-like receptor (TLR) ligand, histamine, peroxisome proliferator-activated receptor ligand, CD14 ligand, CD36 ligand, CD40 ligand, CD68 ligand, integrin β₁, β₂, β₃, or β₅ ligand, integrin αᵥβ₃ ligand, scavenger receptor ligand, phosphatidyl serine receptor ligand, β₂-glycoprotein I (β₂GP1) receptor ligand, scavenger receptor A (SR-A) ligand, macrophage receptor with collagenous structure (MARCO) ligand, scavenger receptor B1 (SR-B1) ligand, LOX-1 ligand, scavenger receptor that binds phosphatidylserine and oxidized lipoprotein (SR-PSOX) ligand, complement component C1q receptor ligand, complement component iC3b receptor ligand, lectin ligand, receptor activator of nuclear factor-κB ligand, CXCR1 ligand, CXCR2 ligand, CXCR3 ligand, CXCR4 ligand, CXCR5 ligand, CXCR6 ligand, CCR1 ligand, CCR2 ligand, CCR3 ligand, CCR4 ligand, CCR5 ligand, CCR6 ligand, CCR7 ligand, CCR8 ligand, CCR9 ligand, CX₃CR1 ligand, XCR1 ligand, PPARγ ligand, Galectin-3 ligand, molecule present at the surface apoptotic cells or secreted by them, any other molecule that can modulate dendritic cell, monocyte, osteoclast, or macrophage gene expression and result in the modification of cellular phenotype, interferon-γ (IFN-γ), interleukin 1 (IL-1), IL-2, IL-3, IL-4 IL-5, IL-6, IL-7, IL-8, IL-9, IL-10, IL-11, IL-12, IL-13, IL-14, IL-15, IL-16, IL-17, IL-18, IL-19, IL-20, IL-21, tumor necrosis factor α (TNF-α), transforming growth factor β (TGF-β), macrophage inflammatory protein 1α (MIP-1α), MIP-1β, MIP-2, MIP-3α, MIP-3β, SLC, I-309, TECK, fractalkine, lymphotactin, MCP-1α, MCP-1β, MCP-2, MCP-3, Eotaxin, MDC, TARC, phosphatidyl serine, GRO-α, ENA-78, NAP-2, IFN-γ-inducible protein 10 (IP-10), Mig, IFN-inducible T-cell alpha chemoattractant (I-TAC), stromal cell derived factor 1 (SDF-1), BCA-1, Bonzo, RANTES, ICAM-3, lysophosphatidyl choline, annexin I, β₂GP1, thrombospondin (TSP), oxidized low density lipoprotein (oxLDL), acetylated LDL, high density lipoprotein (HDL), advanced glycation endpoint LDL, milk fat globule protein (MFG), complement component iC3b, complement component C1q, granulocyte macrophage-colony stimulating factor (GM-CSF), macrophage-colony stimulating factor (M-CSF), apolipoprotein E (apoE), CD154, 12/15 lipoxygenase, Trance, and a fragment, derivative, or mimetic of such or any other molecule that can modulate dendritic cell, monocyte, osteoclast, or macrophage gene expression and a receptor for a ligand that binds one of the effectors selected from the group consisting of interferon-γ (IFN-γ), interleukin 1 (IL-1), IL-2, IL-3, IL-4 IL-5, IL-6, IL-7, IL-8, IL-9, IL-10, IL-11, IL-12, tumor necrosis factor α (TNF-α), transforming growth factor β (TGF-β), macrophage inflammatory protein 1α (MIP-1α), MIP-1β, MIP-2, MIP-3α, MIP-3β, SLC, I-309, TECK, fractalkine, lymphotactin, MCP-1α, MCP-1β, MCP-2, MCP-3, Eotaxin, MDC, TARC, phosphatidyl serine, GRO-α, ENA-78, NAP-2, IFN-γ-inducible protein 10 (IP-10), Mig, IFN-inducible T-cell alpha chemoattractant (I-TAC), stromal cell derived factor 1 (SDF-1), BCA-1, Bonzo, RANTES, ICAM-3, lysophosphatidyl choline, annexin I, β₂GP1, thrombospondin (TSP), oxidized low density lipoprotein (oxLDL), acetylated LDL, high density lipoprotein (HDL), advanced glycation endpoint LDL, milk fat globule protein (MFG), complement component iC3b, complement component C1q, granulocyte macrophage-colony stimulating factor (GM-CSF), macrophage-colony stimulating factor (M-CSF), apolipoprotein E (apoE), CD154, 12/15 lipoxygenase, Trance, and a fragment, derivative, or mimetic of such or any other molecule that can modulate dendritic cell, monocyte, osteoclast, or macrophage gene expression, CD11b, CD14, CD68, FcγR, MHC-II, esterase, ostepontin, IFN-γ, TNF-α, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, MIP-1, MIP-2, MIP-3, MCP-1, MCP-2, MCP-3, brain-derived neurotrophic factor, TRAP, calcitonin receptor, tissue plasminogen activator (tPA), urokinase plasminogen activator (uPA), matrix metalloprotease 1 (MMP-1), MMP-2, MMP-3, MMP-7, MMP-8, MMP-9, MMP-10, MMP-11, MMP-12, MMP-13, MMP-14, MMP-15, MMP-16, MMP-17, MMP-18, MMP-19, MMP-20, MMP-21, MMP-22, MMP-23, MMP-24, MMP-25, MMP-26, MMP-27, MMP-28, SR-A, MARCO, 12/15 lipoxygenase, CD36, SR-B1, CD68, LOX-1, SR-PSOX, Galectin-3, fibroblast growth factors (FGFs), vascular endothelial growth factors (VEGF), platelet-derived growth factor (PDGF), and cathepsin K or is a ligand for the same target as the effector.

10. The method of claim 9, wherein the targeting domain(s) is a ligand for a moiety found on the surface of all cells in the body or only a subset of cells in the body.

11. The method of claim 10, wherein the subset of cells is contained in a particular tissue.

12. The method of claim 1, wherein the targeting domain(s) and the effector domain(s) are part of a fusion protein, or are linked together by a permanent or a transient linkage.

13. The method of claim 1, wherein the dendritic cell, monocyte, osteoclast, or macrophage phenotype that is modified is selected from the group consisting of pro-inflammatory, anti-inflammatory, immunogenic, tolerogenic, tissue-destructive, tissue restorative, cytotoxic, migratory, bone-resorbing, pro-angiogenic, anti-angiogenic, suppressor, antigen presentation, or phagocytic.

14. The method of claim 1, wherein the route of administration to the subject is oral, intravenous, intramuscular, intranasal, inhalatory, intraocular, intrathecal, intradermal, intraperitoneal, subcutaneous, intrapleural, intrauterine, rectal, vaginal, topical, intratumor, transdermal, by eye drops, or transmucosal.

15. The method of claim 1, wherein the effector is administered as a soluble monomer, as part of an oligomer, as part of a dendrimer, as part of a liposome, or by administering a genetic vector that results in the production of the effector in vivo.

16. The method of claim 1, wherein admininistering the effector treats a disease or condition from which the subject is suffering or is at risk of suffering.

17. The method of claim 16, wherein the disease or condition is selected from the group consisting of: arthritis, macular degeneration, cancer, atherosclerosis, osteoporosis, immune inflammation, non-immune inflammation, chronic obstructive pulmonary disease (COPD), tuberculosis, multiple sclerosis, and Alzheimer's disease.

18. The method of claim 1, wherein the route of administration to the subject is oral, intravenous, intramuscular, intranasal, inhalatory, intraocular, intrathecal, intradermal, intraperitoneal, subcutaneous, intrapleural, intrauterine, rectal, vaginal, topical, intratumor, transdermal, by eye drops, or transmucosal.

19. A method of modifying a phenotypic characteristic or phenotype of a macrophage-related cell in vitro comprising contacting the cell with an effector agent selected from the group consisting of: a cytokine, chemokine, pattern recognition receptor ligand, hormone, adrenergic and cholinergic agonist, fatty acid, phospholipid, immunoglobulin, Fc domain of immunoglobulins, lipopolysaccharide (LPS), toll-like receptor (TLR) ligand, histamine, peroxisome proliferator-activated receptor ligand, CD14 ligand, CD36 ligand, CD40 ligand, CD68 ligand, integrin β₁, β₂, β₃, or β₅ ligand, integrin αᵥβ₃ ligand, scavenger receptor ligand, phosphatidyl serine receptor ligand, β₂-glycoprotein I (β₂GP1) receptor ligand, scavenger receptor A (SR-A) ligand, macrophage receptor with collagenous structure (MARCO) ligand, scavenger receptor B1 (SR-B1) ligand, LOX-1 ligand, scavenger receptor that binds phosphatidylserine and oxidized lipoprotein (SR-PSOX) ligand, complement component C1q receptor ligand, complement component iC3b receptor ligand, lectin ligand, receptor activator of nuclear factor-κB ligand, CXCR1 ligand, CXCR2 ligand, CXCR3 ligand, CXCR4 ligand, CXCR5 ligand, CXCR6 ligand, CCR1 ligand, CCR2 ligand, CCR3 ligand, CCR4 ligand, CCR5 ligand, CCR6 ligand, CCR7 ligand, CCR8 ligand, CCR9 ligand, CX₃CR1 ligand, XCR1 ligand, PPARγ ligand, Galectin-3 ligand, a molecule present at the surface apoptotic cells or secreted by them, interferon-γ (IFN-γ), interleukin 1 (IL-1), IL-2, IL-3, IL-4 IL-5, IL-6, IL-7, IL-8, IL-9, IL-10, IL-11, IL-12, tumor necrosis factor α (TNF-α), transforming growth factor β (TGF-β), macrophage inflammatory protein 1α (MIP-1α), MIP-1β, MIP-2, MIP-3α, MIP-3β, SLC, I-309, TECK, fractalkine, lymphotactin, MCP-1α, MCP-1β, MCP-2, MCP-3, Eotaxin, MDC, TARC, phosphatidyl serine, GRO-α, ENA-78, NAP-2, IFN-γ-inducible protein 10 (IP-10), Mig, IFN-inducible T-cell alpha chemoattractant (I-TAC), stromal cell derived factor 1 (SDF-1), BCA-1, Bonzo, RANTES, ICAM-3, lysophosphatidyl choline, annexin I, β₂GP1, thrombospondin (TSP), oxidized low density lipoprotein (oxLDL), acetylated LDL, high density lipoprotein (HDL), advanced glycation endpoint LDL, milk fat globule protein (MFG), complement component iC3b, complement component C1q, granulocyte macrophage-colony stimulating factor (GM-CSF), macrophage-colony stimulating factor (M-CSF), apolipoprotein E (apoE), CD154, 12/15 lipoxygenase, Trance, and a fragment, derivative, or mimetic of such; or (ii) contacting the cell with an effector agent that modifies the expression level of a factor selected from the group consisting of CD11b, CD14, CD68, FcγR, MHC-II, esterase, osteopontin, IFN-γ, TNF-α, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, MIP-1, MIP-2, MIP-3, MCP-1, MCP-2, MCP-3, brain-derived neurotrophic factor, TRAP, calcitonin receptor, tissue plasminogen activator (tPA), urokinase plasminogen activator (uPA), matrix metalloprotease 1 (MMP-1), MMP-2, MMP-3, MMP-7, MMP-8, MMP-9, MMP-10, MMP-11, MMP-12, MMP-13, MMP-14, MMP-15, MMP-16, MMP-17, MMP-18, MMP-19, MMP-20, MMP-21, MMP-22, MMP-23, MMP-24, MMP-25, MMP-26, MMP-27, MMP-28, SR-A, MARCO, 12/15 lipoxygenase, CD36, SR-B1, CD68, LOX-1, SR-PSOX, Galectin-3, fibroblast growth factors (FGFs), vascular endothelial growth factors (VEGF), platelet-derived growth factor (PDGF), and cathepsin K.

20. The method of claim 19, wherein the cell is a macrophage, monocyte, dendritic cell, or osteoclast.
